(19) United States
(12) Patent Application Publication
(10) Pub. No.: US 2007/0060950 A1
(43) Pub. Date: Mar. 15, 2007

(54) Apparatus and methods for delivering sealing materials during a percutaneous procedure to facilitate hemostasis

(76) Inventors: Farhad Khosravi, Los Altos Hills, CA (US); Suresh S. Pai, Mountain View, CA (US); Celso J. Bagaoisan, Union City, CA (US); Scott R. Sershen, Belmont, CA (US)

Correspondence Address: Vista IP Law Group LLP, 2040 MAIN STREET, 9TH FLOOR, IRVINE, CA, 92614, US

(21) Appl. No.: 10/975,205

(22) Filed: Oct. 27, 2004

Related U.S. Application Data
(63) Continuation-in-part of application No. 10/745,946, filed on Dec. 24, 2003.

(51) Int. Cl. A61B 17/08

(52) U.S. Cl. 606/213

(57) ABSTRACT

An apparatus for sealing a puncture extending through tissue to a blood vessel includes a guidewire including an expandable tamp, a retaining sheath for covering the tamp, and a delivery sheath including a primary lumen for receiving the guidewire therethrough and a secondary lumen for delivering sealing compound into the puncture. The guidewire is advanced into the puncture with the tamp in a contracted condition, the tamp is expanded within the vessel after retracting the retaining sheath, and the guidewire is retracted to seal the puncture from the vessel. The delivery sheath is advanced over the guidewire, and sealing compound is introduced into the puncture through the delivery sheath. After introducing the sealing compound, the delivery sheath is removed, and an introducer sheath is advanced over the guidewire to provide access to perform a medical procedure via the vessel after removing the guidewire.

### RELATED APPLICATION DATA

This application is a continuation-in-part of application Ser. No. 10/745,946, filed Dec. 24, 2003, the entire disclosure of which is expressly incorporated herein by reference.

### FIELD OF INVENTION

The present invention relates generally to systems and methods for sealing punctures in a body, and, more particularly, to systems and methods for facilitating hemostasis of a vascular puncture extending through tissue into a blood vessel.

### BACKGROUND

Apparatus and methods are known for accessing a patient's vasculature percutaneously for performing a procedure within the vasculature, and for sealing the puncture that results after completing the procedure. For example, a hollow needle may be inserted through a patient's skin and overlying tissue into a blood vessel. A guidewire is then passed through the needle into the blood vessel, whereupon the needle is removed. An introducer sheath is then advanced over the guidewire into the vessel, e.g., in conjunction with or subsequent to one or more dilators. A catheter or other device may be advanced through the introducer sheath and over the guidewire into a position for performing a medical procedure within the patient's body. In this manner, the introducer sheath facilitates introducing various instruments into the vessel, while minimizing trauma to the vessel wall and blood loss.

Upon completing the procedure, the instrument(s) and introducer sheath are removed, leaving a puncture extending between the skin and the vessel. To seal the puncture, external pressure may be applied to the overlying tissue, e.g., manually and/or using sandbags, until hemostasis occurs. This procedure, however, can be time consuming and expensive, requiring as much as an hour of a medical professional's time. It is also uncomfortable for the patient, and may require the patient to remain immobilized in an operating room, catheter lab, or holding area. In addition, a risk of hematoma exists from bleeding before hemostasis occurs.

Various apparatus and methods have been suggested for sealing a percutaneous puncture instead of or in addition to using external pressure. For example, U.S. Pat. No. 5,108,421 to Fowler discloses using a collagen plug that is delivered into a puncture through tissue. After completing the procedure, the introducer sheath and/or guidewire used to access the patient's vasculature via the puncture are removed. In one embodiment, a catheter is inserted through the puncture into the blood vessel. A balloon on the catheter is expanded and then retracted until the balloon is disposed adjacent the puncture at the wall of the vessel. A plug is then advanced into the puncture until the plug contacts the balloon, thereby preventing the plug from entering the vessel. Once the plug is positioned within the puncture, the balloon is deflated and withdrawn, leaving the plug to expand and seal the puncture and/or promote hemostasis.

By way of another example, U.S. Pat. Nos. 5,192,302 and 5,222,974 issued to Kensey et al. describe using a biodegradable collagen plug that may be delivered through an introducer sheath into a puncture site.

Such sealing methods generally involve introducing plugs or other materials into the puncture after completing the procedure and after removing the introducer sheath. With the introducer sheath removed, there is substantial risk of hematoma within the tissue surrounding the puncture as blood from the vessel leaks into the puncture, which may be uncomfortable and/or harmful to the patient. Further, temporary hemostasis devices for isolating the vessel from the puncture may be difficult to use effectively and/or may be expensive. Despite attempts to isolate the vessel from the puncture while delivering a plug or other sealing material, the sealing material may still leak and/or become exposed in the vessel, where the sealing material risk creating embolic material in the vessel that may harm the patient.

Accordingly, improved systems and methods for sealing punctures, e.g., a percutaneous puncture communicating with a blood vessel, would be useful.

### SUMMARY OF THE INVENTION

The present invention is directed to apparatus, systems, and methods for sealing punctures in a body, and more particularly, to systems and methods for facilitating hemostasis of a vascular puncture extending through tissue into a blood vessel. In various embodiments, the present invention includes systems and methods for delivering a hydrogel or other sealing compound into a percutaneous puncture extending from a patient's skin to a blood vessel or other body lumen before or while performing a vascular procedure to facilitate sealing the puncture after the procedure.

In accordance with one embodiment of the invention, a method is provided for sealing a puncture extending through intervening tissue to a body lumen. In particular, before performing and/or completing a medical procedure via the puncture, a sealing compound may be introduced into the puncture, e.g., to “pre-seal” the puncture. In exemplary embodiments, the sealing compound is a hydrogel, e.g., in a liquid, powder, solid, lyophilized, and/or other dehydrated form. In addition or alternatively, the sealing compound may include one or more synthetic materials, and/or biological pro-thrombotics, such as thrombin, collagen, or other protein-based material, and/or bioabsorbable material, e.g., in a solid, paste, or liquid form. Optionally, the sealing compound may also include therapeutic and/or pharmaceutical agents, e.g., to promote healing, prevent infection and/or other adverse medical events. In one embodiment, the sealing compound includes liquid precursor polymer components delivered from one or more syringes to create a hydrogel in-situ within the puncture. Thus, as used herein “sealing compound” or “sealant” may include any agent, material, or device that may aid in achieving a substantially fluid-tight barrier and/or hemostasis within an organ or tissue that is at risk of post-surgical leakage.

In an exemplary embodiment, a delivery sheath (e.g., an introducer sheath, catheter, or other tubular member), or other delivery device may be introduced into the puncture, and the delivery sheath may be positioned such that a distal end of the delivery sheath is disposed within the intervening tissue. The sealing compound is then delivered through the delivery sheath such that the sealing compound at least partially fills the puncture extra-vascularly and/or permeates into the intervening tissue surrounding the puncture.

For example, the delivery sheath may be advanced over a needle used to create the puncture. The needle is inserted into the patient's skin, through the intervening tissue, and into a blood vessel or other body lumen, e.g., using known methods, followed by insertion of the guidewire. With the needle extending into the body lumen (e.g., blood vessel), the sealing compound may be injected through the delivery sheath into the extra-vascular space and/or puncture track, the needle preventing any substantial amount of the sealing compound from entering the body lumen by providing a temporary seal at the arteriotomy. Alternatively, a catheter or other device may be advanced over the needle and/or over a guidewire advanced through the needle into the body lumen to temporarily seal the body lumen from the puncture. Once the sealing compound is delivered into the puncture, the needle, delivery sheath, and/or catheter may be removed, leaving the guidewire in place. An introducer sheath (which may be the same or different than the tubular member used to deliver the sealing compound) may be introduced and advanced until its distal end enters the body lumen.

After delivering the sealing compound, the body lumen may be accessed via the introducer sheath to perform a medical procedure via the body lumen. For example, one or more instruments may be introduced through the introducer sheath into the body lumen to perform one or more intravascular procedures (e.g., diagnostic and/or therapeutic procedures) within the patient's vasculature.

After completing the procedures( ), the introducer sheath is withdrawn from the puncture, and the sealing compound residing in the extra-vascular tissue space proximate the body lumen and/or within the puncture site may at least partially occlude the puncture to facilitate sealing and/or hemostasis.

In an additional embodiment, for example, the delivery device for the sealing compound may be a needle through which the sealing compound may be injected into the tissue space posterior to the skin and anterior to the body lumen (e.g. blood vessel) to be accessed, such that the sealing compound at least partially fills the space and/or permeates into the intervening tissue surrounding the body lumen. Subsequently, an access needle may be inserted into the patient's skin, through the intervening tissue treated with the sealing compound, and into a blood vessel or other body lumen, followed by the insertion of the guidewire. The access needle may then be removed, leaving the guidewire in place. An introducer sheath may then be introduced and advanced over the guidewire until it enters the body lumen.

After delivering the introducer sheath, the body lumen may be accessed via the introducer sheath to perform a medical procedure via the body lumen. After completing the procedure(s), the introducer sheath may be withdrawn from the puncture, leaving the sealing compound residing in the tissue space proximate the body lumen and/or within the puncture site at least partially occluding the puncture, e.g., to facilitate sealing and/or hemostasis.

In some embodiments, the sealing compound, e.g., a lyophilized hydrogel, may be exposed to bodily fluids from the body lumen, whereupon the sealing compound may hydrate and/or expand to facilitate hemostasis within the puncture when the introducer sheath is withdrawn after the procedure. Optionally, external pressure may be applied to the intervening tissue to enhance hemostasis within the puncture and/or another sealing compound may be delivered into the puncture.

In another embodiment of the invention, an apparatus is provided for sealing a puncture extending through tissue, the apparatus including an elongate tubular member having a proximal end, a distal end terminating in a distal tip sized and/or shaped for insertion into the puncture, and a lumen extending between the proximal and distal ends. A sealing compound is carried on an exterior of the tubular member proximal the distal tip such that the sealing compound is disposed within the puncture when the tubular member is introduced into the puncture. By way of example, the sealing compound may include a hydrogel.

Optionally, a cover may extend along the exterior of the tubular member such that the cover covers the sealing compound, the cover being at least partially removable to expose the sealing compound. A lubricious coating may be provided on the exterior of the tubular member, and the sealing compound may overly the lubricious coating to facilitate the tubular member being slidable, e.g., proximally, relative to the sealing compound.

In another embodiment, a method for sealing a puncture extending through tissue is provided, wherein a tubular member, e.g., a delivery sheath and/or introducer sheath, carrying a sealing compound on an exterior thereof is introduced into the puncture. A body lumen is accessed through the puncture, e.g., through the tubular member or a separate introducer sheath, to perform a medical procedure via the body lumen. When the introducer sheath is withdrawn from the puncture, i.e., after completing the medical procedure, the sealing compound remains within the puncture to at least partially seal the puncture. For example, the sealing compound may be a lyophilized hydrogel that, when exposed to bodily fluids from the body lumen, expands to facilitate hemostasis within the puncture.

In accordance with still another embodiment, a system is provided for sealing a puncture extending through tissue to a body lumen of a patient that includes a guidewire or other elongate member including proximal and distal ends, and an expandable member or “tamp” on the distal end that is expandable from a contracted condition to an enlarged condition. The system may also include a delivery sheath including a proximal end, a distal end sized for insertion through the puncture, a primary lumen extending between the delivery sheath proximal and distal ends for receiving the elongate member therethrough with the expandable member in the contracted condition, and a secondary lumen extending from a side port in the delivery sheath proximal end to one or more outlets located between the delivery sheath proximal and distal ends for delivering a sealing compound into the puncture.

In one embodiment, the expandable member is biased towards the enlarged condition, and the system includes a removable constraint for maintaining the expandable member in the contracted condition. For example, the constraint may include a tubular member slidable over the elongate member, e.g., to cover the expandable member (e.g., to maintain the expandable member in the contracted condition), and uncover the expandable member (e.g., to allow the expandable member to expand towards the enlarged condition). Alternatively, the expandable member may be selectively expandable, e.g., by activating an actuator on a proximal end of the elongate member. In exemplary embodiments, the expandable may be a mesh structure, an expandable frame, and the like, e.g., including a substantially nonporous covering on at least a portion thereof.

In another embodiment, a method is provided for sealing a puncture extending through tissue to a body lumen of a patient that includes placing a guidewire or other elongate member through the puncture into the body lumen, inserting a proximal portion of the guidewire into a primary lumen of a delivery sheath; and introducing the delivery sheath into the puncture by advancing the delivery sheath over the guidewire with the guidewire sliding within the primary lumen. A sealing compound may be introduced into the puncture through one or more secondary lumens of the delivery sheath. After introducing the sealing compound, the body lumen may be accessed, e.g., using an introducer sheath advanced over the guidewire, through the puncture to perform a medical procedure via the body lumen.

In yet another embodiment, a method is provided for sealing a puncture extending through tissue to a body lumen of a patient that includes advancing a guidewire or other elongate member carrying an expandable member in a contracted condition through the puncture until the expandable member is disposed within the body lumen, expanding the expandable member within the body lumen, and retracting the elongate member until the expanded expandable member substantially seals the puncture from the body lumen. A delivery sheath may be advanced into the puncture, e.g., over the elongate member, and sealing compound may be introduced into the puncture through the delivery sheath. After introducing the sealing compound, the body lumen may be accessed through the puncture to perform a medical procedure via the body lumen, e.g., after removing the elongate member from the puncture.

Other aspects and features of the invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of a system for sealing a puncture, including a delivery sheath, a needle, a guidewire, and a syringe assembly for delivering sealing compound.

FIGS. 2A-2D are cross-sectional views of a patient's body, illustrating exemplary methods for sealing a puncture extending between the patient's skin and a blood vessel.

FIG. 3 is a cross-sectional view of a patient's body, illustrating another exemplary method for sealing a puncture extending between the patient's skin and a blood vessel.

FIGS. 4A and 4B are cross-sectional views of a patient's body illustrating yet another exemplary method for sealing a puncture extending between the patient's skin and a blood vessel.

FIGS. 5A and 5B are cross-sectional views of a patient's body illustrating still another exemplary method for sealing a puncture extending between the patient's skin and a blood vessel.

FIG. 6 is a cross-sectional side view of another apparatus for sealing a puncture.

FIG. 7 is a side view of another embodiment of a delivery sheath for delivering sealing compound into a puncture extending through tissue.

FIGS. 8A and 8B are cross-sectional views of a patient's body, illustrating a method for sealing a puncture extending between the patient's skin and a blood vessel, using the delivery sheath of FIG. 7.

FIGS. 9A-9C are side views of components of another embodiment of an apparatus for sealing a puncture extending through tissue, including a delivery sheath, a retaining sheath, and a guidewire with self-expanding tamp, respectively.

FIGS. 10A and 10B are side views of a retaining sheath received over a guidewire with an expandable tamp, with the retaining sheath retracted and extended such that the tamp is in contracted and enlarged conditions, respectively.

FIGS. 11A-11E are cross-sectional views of a patient's body, illustrating a method for sealing a puncture extending between the patient's skin and a blood vessel, using the system of FIGS. 9A-9C.

FIGS. 12A and 12B are side views of another embodiment of a guidewire with an expandable tamp in contracted and enlarged conditions, respectively.

### DETAILED DESCRIPTION OF THE ILLUSTRATED EMBODIMENTS

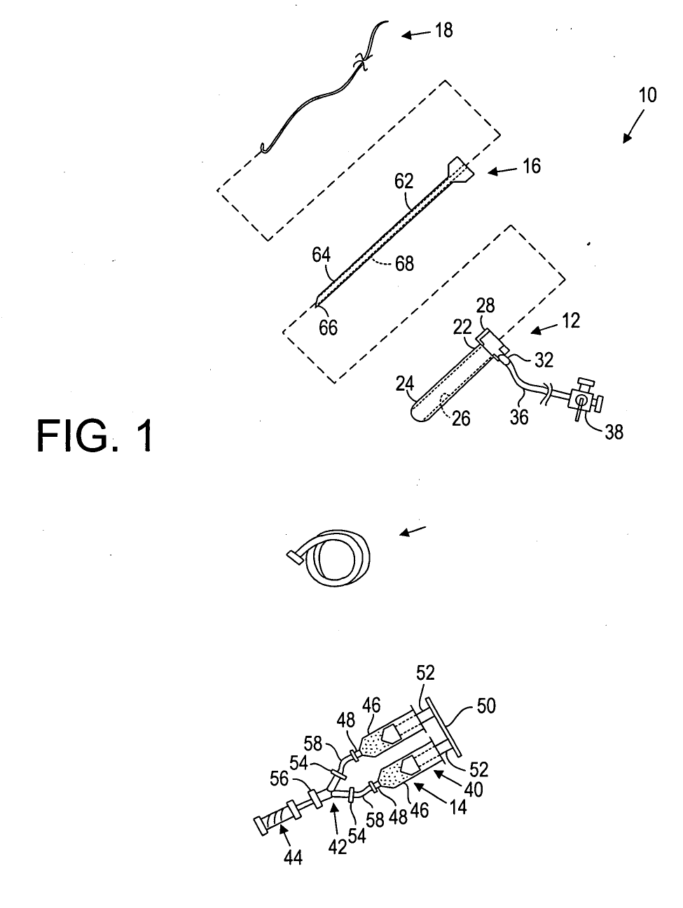

Turning to the drawings, FIG. 1 shows an exemplary embodiment of a system 10 for sealing a puncture through tissue, e.g., a percutaneous puncture for accessing an artery or other blood vessel (not shown). Generally, the system 10 includes a delivery sheath 12 and a delivery device 14 for delivering a sealing compound into the puncture. In the illustrated embodiment, the system 10 includes other components, including a needle 16 for creating the puncture, a guidewire 18, and tubing 20. In addition or alternatively, the system 10 may include other or further components for creating the puncture, delivering the delivery sheath 12 and/or guidewire 18 into a body lumen, and/or introducing instruments into the puncture (such as a standard introducer sheath, not shown), as are known to those of ordinary skill in the art.

The delivery sheath 12 generally is an elongate tubular member including a proximal end 22, a distal end 24, and a lumen 26 extending between the proximal and distal ends 22, 24. The delivery sheath 12 terminates in a tapered distal tip 25 for facilitating advancing the delivery sheath 12 substantially atraumatically through tissue into a puncture, as is known to those skilled in the art. Alternatively, the distal end of the delivery sheath 12 may include one or more side outlet ports (not shown) to direct the sealing compound during delivery. Exemplary materials for the delivery sheath 12 may include plastics, such as polyamide, PEEK, nylon, PET, PEBAX, and polyethylene, metals, such as stainless steel, and nickel titanium, and/or composite materials.

A housing 28 may be attached to or otherwise provided on the proximal end 22 of the delivery sheath 12. The housing 28 may include one or more side ports 32 that communicate with an interior of the housing 28 and the lumen 26 of the delivery sheath 12. Preferably, at least one side port 32 is provided that includes a section of flexible tubing 36 terminating in a manual shut-off valve 38 and/or a luer lock or other connector (not shown), e.g., to facilitate connecting tubing 20 and the like to the side port 32. The housing 28 may also include one or more seals (not shown), e.g., a hemostatic seal, for sealing the lumen 26 of the delivery sheath 12, yet accommodating inserting the needle 16 and/or one or more instruments (not shown) into the lumen 26 of the delivery sheath 12 while preventing body fluids, such as blood, from escaping proximally from the delivery sheath 12, as is known in the art.

The delivery device 14 may include a single syringe, or a multiple syringe assembly. As shown in FIG. 1, the delivery device 14 is a dual syringe assembly 40 that includes two components of a sealing compound, a “Y” fitting 42, and a static mixer 44. The syringe assembly 40 includes a pair of syringe barrels 46, including outlets 48 and a plunger assembly 50 slidable into the barrels 46 to cause the components therein to be delivered through the outlets 48. A pair of plungers 52 are coupled to one another and yet are received in respective barrels 46. In this manner, both plungers 52 may be manually depressed substantially simultaneously to deliver the components together from the syringe barrels 46. Alternatively, a system for automatically advancing the plungers 52 and/or otherwise delivering the components in the barrels 50 may be used, such as those disclosed in application Ser. No. 10/806,934, filed Mar. 22, 2004, the entire disclosure of which is expressly incorporated herein by reference.

The “Y” fitting 42 includes proximal sections 54 that communicate with a single distal section 56. In this manner, the “Y” fitting 42 may be connectable to outlets 48 of the syringe barrels 46, e.g., by tubing 58 or directly (not shown), such that the components ejected out of the barrels 46 may mix before being directed into the side port 32 of the delivery sheath 12. The proximal and distal sections 54, 56 may include connectors, e.g., luer lock connectors and the like (not shown), for connecting with the outlets 48 of the syringes 46 and/or with the mixer 44, tubing 20, 58, and/or the side port 32 of the introducer sheath assembly 12. The mixer 44 may be a tubular body including vanes or other internal structures (not shown) that enhance the components mixing thoroughly together as they pass therethrough. Similar to the “Y” fitting 42, the mixer 44 may include connectors (not shown) for releasably or substantially permanently connecting the mixer 44 to the “Y” fitting 42, tubing 20, and the like.

Respective precursor polymer components may be provided in each syringe barrel 46 of the syringe assembly 40 that, when mixed together, are activated to form a hydrogel. Additional information on such hydrogels and systems for delivering them are disclosed in U.S. Pat. Nos. 6,152,943, 6,165,201, 6,179,862, 6,514,534, 6,379,373, and 6,703,047 and in co-pending application Ser. Nos. 10/010,715 filed Nov. 9, 2001, Ser. No. 10/068,807 filed Feb. 5, 2002, and Ser. No. 10/454,362, filed Jun. 4, 2003. The disclosures of these references and any others cited therein are expressly incorporated by reference herein.

In the illustrated embodiment, the system 10 includes a needle 16 to facilitate inserting the delivery sheath 12 through tissue. The needle 16 may be a substantially rigid elongate tube, e.g., made from stainless steel and the like, including a proximal portion 62, a distal portion 64 terminating in a beveled or otherwise sharpened distal tip 66, and a lumen 68 extending between the proximal and distal portions 62, 64. The proximal portion 62 of the needle 16 may include one or more seals, e.g., similar to the housing 28 on the delivery sheath 12, to facilitate inserting an instrument, such as guidewire 18, through the lumen 68 while substantially sealing the needle 16 from fluid flow therethrough. The guidewire 18 may include one or more known guidewires, e.g., including a “J” tip and the like, as is well known in the art.

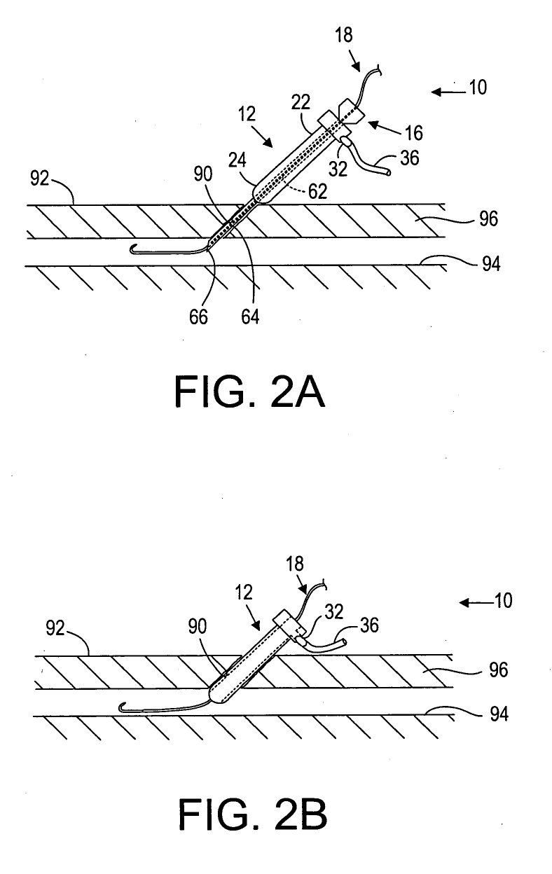

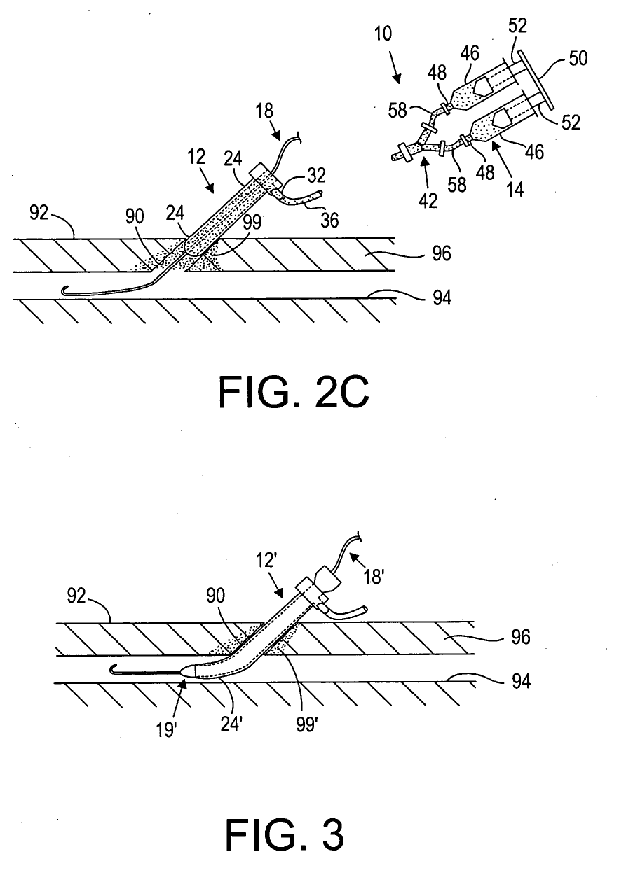

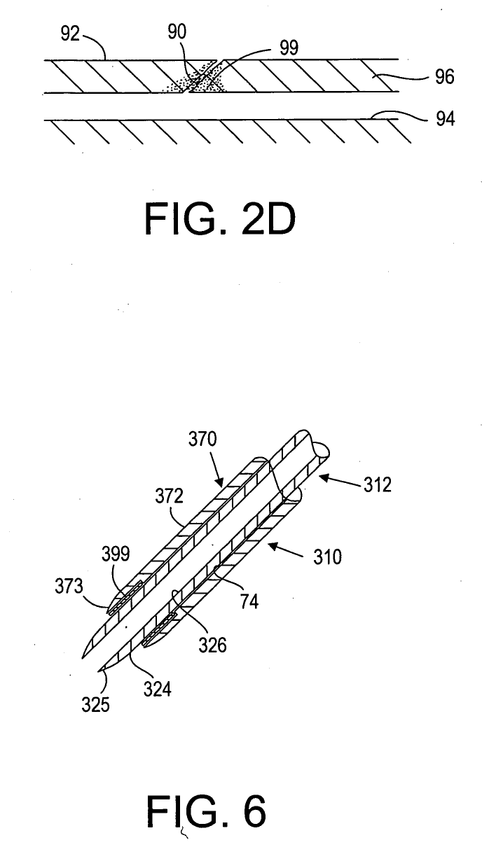

Turning to FIGS. 2A-2D, an exemplary method for sealing a passage through tissue is shown, e.g., using the system 10 of FIG. 1. In the illustrated embodiment, the passage is a percutaneous puncture 90 extending from a patient's skin 92 to a blood vessel or other body lumen 94. For example, the vessel 94 may be a peripheral artery, e.g., a femoral artery, a carotid artery, and the like. It will be appreciated that systems and methods constructed and undertaken in accordance with various embodiments of the invention may be used to seal other passages through tissue within a patient's body.

Initially, as shown in FIG. 2A, the delivery sheath 12 may be introduced into the puncture 90 such that the distal end 24 of the delivery sheath 12 is disposed within the vessel 94. For example, the delivery sheath 12 may be disposed over the proximal portion 62 of the needle 16 such that the distal end 24 of the delivery sheath 12 is located proximal to a distal portion 64 of the needle 16. The sharpened distal tip 66 of the needle 16 may be inserted into the patient's skin 92, and through any intervening tissue 96 into the vessel 94, thereby creating the puncture 90. Once the distal tip 66 is positioned within the vessel 94, the delivery sheath 12 may be advanced distally over the needle 16 into the puncture 90 until the distal tip 26 enters the vessel 94. The guidewire 18 may be advanced through the needle 16 into the vessel 94 either before or after the delivery sheath 12 is advanced into the puncture 90.

Alternatively, a hollow needle, similar to needle 16, may be inserted through a patient's skin and intervening tissue into a blood vessel without the delivery sheath 12. A guidewire, similar to guidewire 18, may be passed through a lumen of the needle into the vessel 94, whereupon the needle may be removed. The delivery sheath 12 may then be advanced over the guidewire into the vessel 94, e.g., in conjunction with or subsequent to one or more tubular dilators (not shown). It will be appreciated by those skilled in the art that the delivery sheath 12 may be introduced into the puncture 90 using other conventional methods known for introducing introducer sheaths through intervening tissue into a blood vessel.

As shown in FIG. 2B, once the delivery sheath 12 and guidewire 18 are positioned in the vessel 94, the needle 16 may be removed from the puncture 90, leaving the delivery sheath 12 and guidewire 18 in place. Then, as shown in FIG. 2C, the delivery sheath 12 may be partially withdrawn from the puncture 90 until the distal end 24 of the delivery sheath 12 is located proximal to the vessel 94, i.e., within the intervening tissue 96.

Optionally, the side port 32 may be used as a bleed back port to assist positioning the delivery sheath 12 in the puncture 90. For example, with the shut-off valve 38 open, blood may flow proximally from the vessel 94 through the delivery sheath 12 and out the side port 32. When the delivery sheath 12 is retracted, the distal end 24 may be withdrawn from the vessel 94, whereupon blood flow out the side port 32 may stop, indicating that the distal end 24 of the delivery sheath 12 is located within the puncture 90. Alternatively, visual markers (not shown) may be provided on the exterior of the delivery sheath 12 that may be used to measure or provide other visual indication that the delivery sheath 12 has been withdrawn sufficiently from the vessel 94.

A sealing compound 99 may then be delivered into the puncture 90, e.g., such that the sealing compound 99 at least partially surrounds the delivery sheath 12 and/or extends towards the vessel 94. In one embodiment, the sealing compound 99 is a liquid or other flowable material that may be injected into the puncture 90 such that the sealing compound 99 permeates into the intervening tissue 96 surrounding the puncture 90. In one embodiment, the sealing compound 99 may include one or multiple component precursor polymers that create a hydrogel when mixed together and/or upon contacting tissue fluids, as described above. Such a hydrogel sealing compound may be particularly useful, because it may be substantially harmless to the patient if it leaks into the vessel 94. Unlike collagen or other hemostasis-promoting materials, appropriately selected hydrogel precursor polymers do not cause thrombosis and/or embolism when exposed to blood. In fact, such precursor polymers, if exposed within a vessel, will simply dilute and flow away, where they may be safely metabolized naturally without substantial risk of creating thrombus.

In one embodiment, a two-part sealing compound is delivered into the puncture 90 using a dual syringe assembly 40, similar to that shown in FIG. 1 and described above. The precursor polymers or other components in the syringe barrels 46 may be mixed or otherwise prepared before the procedure using known methods. For example, the “Y” fitting 42, mixer 44, and/or, tubing 20, 58 may be coupled to one another and/or to the outlets 48 before the procedure or at the time of delivery. Similarly, tubing 20 may be connected to the side port 32 before the procedure or immediately before delivery. Preferably, the tubing 20 is connected to the side port 32 immediately before delivery so that the tubing 20 does not obstruct or otherwise interfere with introducing the delivery sheath 12, needle 16, and/or guidewire 18, as described above.

Once the delivery sheath 12 is coupled to the deliver device 14, the plunger assembly 50 may be manually (or optionally automatically, upon actuation) depressed, advancing the plungers 52 substantially simultaneously into the barrels 46, and delivering the precursor polymers substantially simultaneously from the outlets 48. The precursor polymers mix in the “Y” fitting 42 and mixer 44 into a liquid sealing compound, and are then delivered into the side port 32 of the delivery sheath 12 via tubing 20. The liquid sealing compound 99 exits the distal end 24 of the delivery sheath 12, and enters the puncture 90, where it at least partially surrounds the delivery sheath 12 and/or permeates into the intervening tissue 96.

The sealing compound 99 should be permitted sufficient time to “gel” or cure and/or solidify within the puncture 90, e.g., between about five (5) and one hundred eighty (180) seconds. Once the sealing compound is delivered into the puncture 90 and/or at least partially gelled, the delivery sheath 12 is removed while the vessel 94 is compressed proximally (upstream relative to the vessel 94) to prevent blood from leaking out of the puncture 90. An introducer sheath (not shown), such as those known in the art, may be introduced and advanced over the guidewire 18 until the distal end 24 enters the vessel 94, whereupon the compression is relieved to allow blood flow to resume in the vessel 94.

In further alternatives, the delivery sheath 12 may include one or more secondary lumens (not shown) located in the wall of the delivery sheath 12 that extend from the proximal end 22 to an intermediate location proximal to the distal end 24. One or more side outlets (also not shown) may be provided in the side wall of the delivery sheath 12 and one or more inlet side ports (also not shown) may be provided in the housing 28 that communicate with the secondary lumen(s). For example, if a single secondary lumen is provided, the tubing from the delivery device may be coupled to the inlet side port for delivering the sealing compound via the secondary lumen to the side wall outlet. Alternatively, two secondary lumens may be provided, and each precursor polymer may be delivered into a respective secondary lumen such that precursor polymers mix together when they exit the side outlets within the puncture 90.

One advantage of these alternatives is that the sealing compound may be delivered into the intervening tissue surrounding the puncture 90 without having to retract the delivery sheath 12, thereby reducing handling of the delivery sheath 12. In addition, these alternatives may allow the lumen 28 to remain unobstructed, since the secondary lumen(s) is(are) used to deliver the sealing compound, which may gel or otherwise solidify to obstruct the secondary lumen(s). With the lumen 28 unobstructed by sealing compound, the delivery sheath 12 may be used as an introducer sheath subsequent to delivering the sealing compound, as explained further below.

For example, turning to FIG. 3, a delivery sheath 12,′ including one or more secondary lumens (not shown), is advanced over a needle (not shown), similar to the needle 16 of FIG. 1 until the distal end 24′ enters the puncture 90 but does not enter the vessel 94. The needle 16′ may then be removed, and sealing compound 99′ delivered into the puncture 90 through the one or more secondary lumens. The delivery sheath 12′ may then be advanced over the guidewire 18′ until the distal end 24′ is disposed within the vessel 94, e.g., in conjunction with one or more dilators (an exemplary dilator 19′ being shown in FIG. 3), as is known to those skilled in the art. Alternatively, the needle may remain in the puncture 90 while the sealing compound 99′ is delivered, and removed before or after the delivery sheath 12′ is advanced into the vessel 94. In still another alternative, the delivery sheath 12′ may be removed after delivering the sealing compound 99, leaving the guidewire 18′ in place. An introducer sheath (not shown), in conjunction with one or more dilators (also not shown), may be advanced into the puncture 90 until the distal end of the introducer sheath enters the vessel 94.

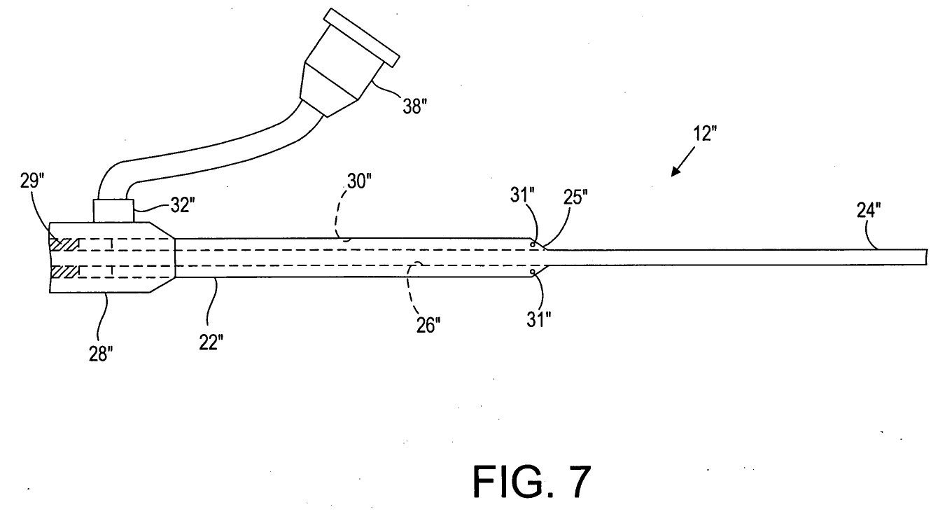

Turning to FIG. 7, another embodiment of a delivery sheath 12″ is shown that may be constructed similar to the embodiments described elsewhere herein. Generally, the delivery sheath 12″ is an elongate tubular member, including a proximal end 22,″ a distal end 24,″ and a primary or guidewire lumen 26″ extending between the proximal and distal ends 22,″ 24.″ The delivery sheath 12″ may terminate in a substantially atraumatic distal tip (not shown), similar to the previous embodiments.

In addition, the delivery sheath 12″ includes one or more secondary or injection lumens 30″ that extend from the proximal end 22″ to one or more outlets 31″ (e.g., two, as shown) in the wall of the delivery sheath 12.″ As shown, a single secondary lumen 30″ is disposed concentrically around the primary lumen 26.″ Alternatively, one or more secondary lumens (not shown) may be formed or otherwise provided in the wall of the delivery sheath 12,″ e.g., in a side-by-side arrangement. As used herein, “primary” and “secondary” may simply mean “first” and “second,” respectively, and may not necessarily require that one lumen be larger or more significant than the other. The primary lumen 26″ may be of sufficient size to accommodate sliding a guidewire therethrough, e.g., between about 0.014 and 0.018 inch (0.35-0.45 mm), while the secondary lumen 30″ may be of sufficient size to accommodate delivering-sealing compound therethrough.

The secondary lumen 30″ extends from housing 28″ to an intermediate portion 25″ between the proximal and distal ends 22,″ 24.″ As shown, the intermediate portion 25″ tapers where the secondary lumen 30″ terminates, with the delivery sheath 12″ having a smaller diameter from the intermediate portion 25″ to the distal end 24″ (e.g., since only the primary lumen 26″ extends along this portion of the delivery sheath 12″). The smaller diameter distal portion may have a desired length, e.g., at least about five millimeters (5 mm). The outlet(s) 31″ may be provided on the intermediate portion 25,″ e.g., where the delivery sheath 12″ tapers, which may facilitate directing the sealing compound delivered through the secondary lumen 30″ radially outwardly away from the delivery sheath 12.″

The housing 28″ may be attached to or otherwise provided on the proximal end 22″ of the delivery sheath 12,″ similar to the previous embodiments. The housing 28″ may include one or more side ports 32″ (one shown) that communicate with an interior of the housing 28″ and the secondary lumen 30″ of the delivery sheath 12.″ The housing 28″ may include one or more seals 29″ to seal the interior of the housing 28″ such that sealing compound delivered from the side port 32″ may be directed through the secondary lumen 30.″ In addition, the housing 28″ may also include one or more seals (not shown), e.g., a hemostatic seal, for sealing the primary lumen 26″ while accommodating inserting a needle or other instrument (not shown) into the lumen 26.″

A section of flexible tubing 36″ may be connected to or otherwise extend from the side port 32″ to a luer lock adapter 38,″ a manual shut-off valve (not shown), and/or other connector (also not shown), e.g., to facilitate connecting tubing 20 and the like to the side port 32. A source of sealing compound, such as the dual-syringe assembly 40 described above, may be connected to the luer lock adapter 38.″

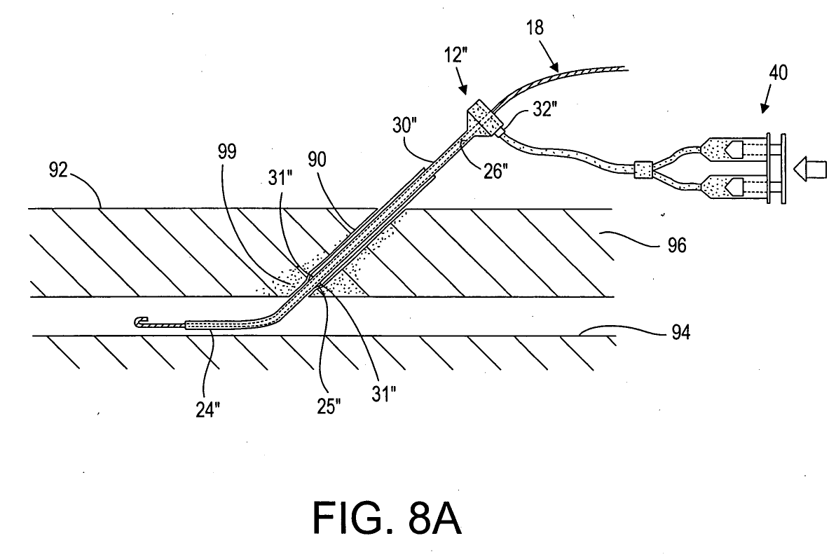

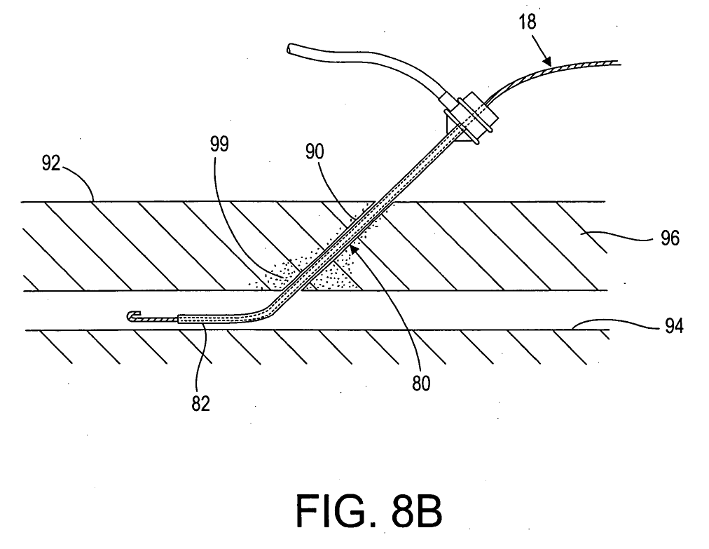

Turning to FIGS. 8A and 8B, a method for sealing a puncture 90 is shown using the delivery sheath 12.″ Similar to the previous embodiments, a guidewire 18 may be placed through the puncture 90 from the patient's skin 92 to the blood vessel 94. For example, a needle (not shown) may be inserted through the patient's skin 92 and intervening tissue 96 into the vessel 94, the guidewire 18 may be advanced through a lumen of the needle, and the needle may then be removed.

Once the guidewire 18 is placed, the guidewire 18 may be backloaded through the primary lumen 26″ of the delivery sheath 12″ and the delivery sheath 12″ may be advanced through the puncture 90 over the guidewire 18 until the distal end 24″ of the delivery sheath 12″ is disposed within the vessel 94. Optionally, the secondary lumen 30″ may be used as a bleed back indicator to facilitate positioning the delivery sheath 12″ relative to the vessel 94. For example, the delivery sheath 12″ may be advanced into the puncture 90 until the outlets 31″ become exposed within the vessel 94, whereupon blood may flow proximally into the outlets 31,″ through the secondary lumen 30,″ and out the side port 32″ to provide a visual indication that the outlets 31″ are adjacent the vessel 94.

The delivery sheath 12″ may then be withdrawn partially to dispose the outlets 31″ adjacent the vessel 94, e.g., at least about five millimeters away from the vessel wall. The dual syringe assembly 40 (or other delivery device) may be connected to the side port 32″ and sealing compound 99 may be delivered through the secondary lumen 30″ and outlets 31″ into the tissue 96 surrounding the puncture 90, similar to the other embodiments described herein. Optionally, the delivery sheath 12″ may be retracted as the sealing compound 99 is delivered.

Once sufficient sealing compound has been delivered into the puncture 90, the delivery sheath 12″ may be removed from the puncture 90. The guidewire 18 may remain, and an introducer sheath 80 may be advanced over the guidewire 18 into the puncture 90 until a distal end 82 of the introducer sheath 80 is disposed within the vessel 94. The guidewire 18 (and any associated dilators, not shown) may be removed, and one or more catheters or other instruments (also not shown) may be advanced through the introducer sheath 80 to perform one or more diagnostic and/or interventional procedures within the patient's body, similar to the embodiments described elsewhere herein.

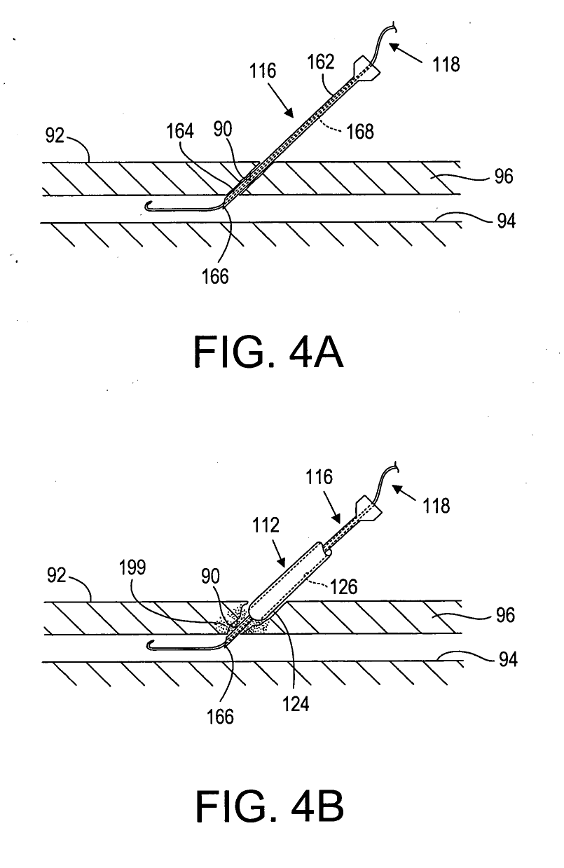

Turning to FIGS. 4A and 4B, another method for pre-sealing a puncture 90 is shown. Similar to the methods described above, a needle 116 may be inserted into tissue 96 to create puncture 90 and advanced until distal tip 166 enters vessel 94, as shown in FIG. 4A. Delivery sheath 112 (which may be similar to any of the embodiments described herein) may be advanced over the needle 116 until distal end 124 of the delivery sheath 112 is disposed proximal to the vessel 94. For example, the delivery sheath 112 may be disposed initially on a proximal portion of the needle 116 when the needle 116 is inserted (not shown), may be advanced over the needle 116 after the puncture 90 is created, or may be advanced together with the needle 116, similar to the embodiments described above.

As shown in FIG. 4B, sealing compound 199 may then be delivered through lumen 126 of the delivery sheath 112 into the puncture 99. In this embodiment, the needle 116 remains in the puncture 90 such that the distal tip 166 extends into the vessel 94, e.g., to at least partially and preferably substantially seal the vessel 94 from the puncture 90. In this manner, the sealing compound 99 may be introduced into the puncture 90 via the delivery sheath 112, with the needle 116 preventing substantial amounts of the sealing compound 99 from entering the vessel 94. This alternative may allow thrombogenic and/or bioabsorbable sealing materials, such as collagen thrombin, fibrin, polyglycolic acids (PGA's), polyactides (PLA's), natural or synthetic tissue adhesives, and the like, to be introduced into the puncture 90 without substantial risk of their entering the vessel 94. Optionally, a catheter or other device (not shown) may be advanced over the needle 116 to further seal the vessel 94 from the puncture 90 before the sealing compound 99 is introduced.

A guidewire 118 may be advanced through the needle 116 before or after delivering the sealing compound 99. After the sealing compound 99 is delivered, the needle 116, delivery sheath 112, and/or catheter may be removed before an introducer sheath (not shown) is introduced into the puncture 90 over the guidewire 118 and advanced into the vessel 94. Alternatively, the delivery sheath 112 may be advanced over the guidewire until the distal end 124 enters the vessel 94, and used as an introducer sheath, e.g., if the delivery sheath includes one or more secondary lumens through which the sealing compound 99 is delivered. Thus, the sealing compound 99 may “pre-seal” the puncture 90, e.g., bulking the puncture 90 and/or creating a pillowing effect that may enhance sealing of the puncture 90, as described further below.

Alternatively, the sealing compound may be delivered directly through a needle, e.g., the needle used to create the puncture, without using a separate delivery sheath. For example, similar to FIG. 4A, the needle 116 may be inserted into tissue 96 to create puncture 90 and advanced until distal tip 166 of the needle 116 enters vessel 94. The user may confirm that the distal tip 166 has entered the vessel 94 simply by “bleed back,” i.e., by internal blood pressure within the vessel 94 forcing a small amount of blood proximally through lumen 168 of the needle 116 until it visibly escapes from proximal end 162 of the needle 116.

A guidewire 118 may be inserted into the proximal end 162 of the needle 116 and advanced through the lumen 168 until the guidewire 118 enters the vessel 94. The needle 116 may then be withdrawn partially to withdraw the distal tip 166 out of the vessel 94 a predetermined distance into the puncture 90, e.g., between about five and ten millimeters (5-10 mm).

Optionally, the needle 116 may include an annular washer or other element (not shown) on the exterior of the needle 116 to facilitate withdrawing the distal tip 166 the predetermined distance. The washer may be slidable along the exterior of the needle 116 such that, once the distal tip 116 is disposed within the vessel 94, the washer may be advanced distally along the needle 116 until it contacts the patient's skin 92. The washer may then be maintained against the skin 92 while the needle 116 is withdrawn to provide a visual indication of the distance that the needle 116, and consequently the distal tip 166, are being withdrawn relative to the vessel 94.

In addition or alternatively, the needle 116 may include distance markings (not shown) along at least a portion of the exterior for providing similar visual indication of the distance that the needle 116 is withdrawn. For example, when bleed back occurs, the user may note a marker adjacent the patient's skin 92. The needle 116 may then be withdrawn until one or more markers on the needle 116 become visible, thereby providing a qualitative, or possibly quantitative, distance of withdrawal.

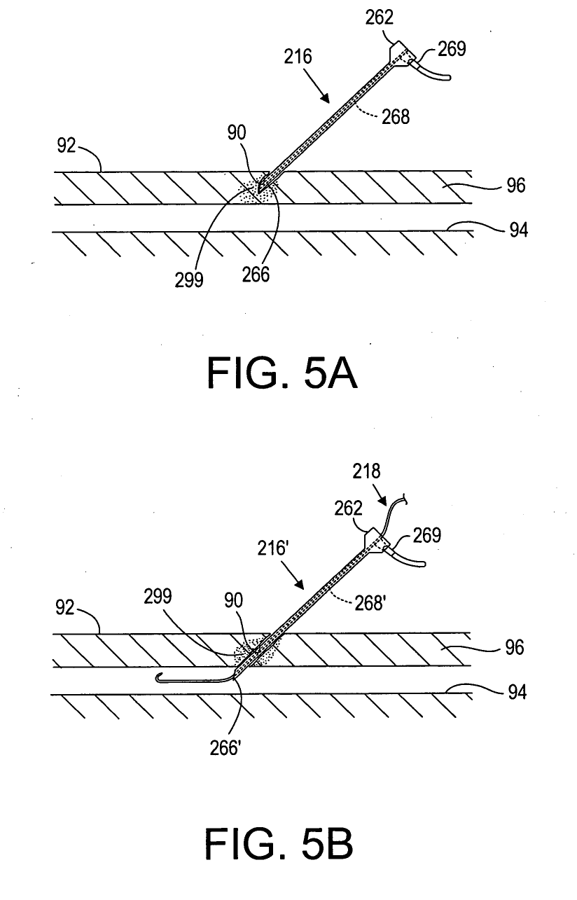

Sealing compound 99 may then be delivered into the puncture 90, except through the lumen 168 of the needle 116 itself, similar to the method described with reference to FIG. 5A below. For example, a fitting (not shown) may be coupled to the proximal end 162 of the needle 116 to allow a source of sealing compound to communicate with the lumen 168 of the needle 116.

In one embodiment, the fitting may be a Touhy-Borst or other “Y” adapter (also not shown), that may include two proximal ends that communicate with a second distal end. One proximal end may include a hemostatic seal and the like, such that the adapter may be advanced over the guidewire 118, e.g., by backloading the guidewire 118 into the distal end of the adapter and out through the proximal end including the hemostatic seal. The other proximal end of the adapter may be connected to a source of sealing compound, e.g., a syringe connected by tubing, similar to the embodiments described above.

In this manner, the sealing compound 99 may be introduced into the puncture 90 from the source of sealing compound, through the adapter and lumen 168 of the needle 168 and out the distal tip 166. Optionally, the needle 116 may be at least partially withdrawn as the sealing compound 99 is delivered to fill the puncture 90 with the sealing compound 99.

Once sufficient sealing compound 99 has been delivered (and, optionally, provided sufficient time to gel or otherwise set-up), the needle 116 may be removed entirely from the puncture 90, leaving the guidewire 118 in place. Optionally, proximal compression on the skin 92 may be applied to pinch or otherwise secure the guidewire 118 while the needle 116 is removed, as may also be performed for other methods described herein. An introducer sheath may then be advanced over the guidewire 118 to access the vessel 94 through the puncture 90 and sealing compound 99, similar to the other embodiments described herein.

Turning to FIGS. 5A and 5B, another method is shown for pre-sealing a puncture 90 through tissue 96, e.g., communicating with vessel 94 or other body lumen. As shown in FIG. 5A, a needle 116, including a sharpened distal tip 266 and a lumen 268, is inserted through the patient's skin 92 into tissue 96 without penetrating into the vessel 94. The needle 116 may include a side port 269 that may be coupled to a delivery device, such as the dual syringe assembly 14 shown in FIG. 1 or other source of sealing compound (not shown in FIG. 5A). Alternatively, a delivery device (not shown) may be inserted into the lumen 268, e.g., through one or more seals (also not shown) at the proximal end 262 of the needle 216.

Sealing compound (such as any of those described herein) may be delivered into the side port 269, through the lumen 268, and out the distal tip 266 of the needle 216 into the extra-vascular space above the vessel 94. Because the wall of the vessel 94 has not been pierced, the sealing compound 299 may fill the puncture 90 and/or permeate into the surrounding tissue 96, thereby bulking the puncture 90 and/or creating a pillowing effect above the vessel 94.

After delivering the sealing compound 299, the needle 216 may be removed, and another needle 216′ (which may be similar to the needle 216) advanced through the tissue 96 and/or through the sealing compound 299 until its distal tip 266′ penetrates the wall of the vessel 94. A guidewire 218 may be advanced through the needle 216′ into the vessel 94, the needle 216′ may be removed from the puncture 90, and an introducer sheath (not shown) may be advanced over the guidewire 218 into the vessel 94. Thus, in this embodiment, there may be no need for a separate delivery sheath or other device to deliver the sealing compound 299. Alternatively, a single needle may be used to deliver the sealing compound 299 and access the vessel 94, e.g., if the needle includes separate lumens for delivering the sealing compound 299 and advancing instruments into the vessel 94, similar to embodiments of the delivery sheath described above.

Once sealing compound is delivered into a puncture to pre-seal the puncture, e.g., using any of the methods described herein, an introducer sheath (which may be the same or different than the delivery sheaths described above) may then be used to access the vessel, e.g., to perform one or more therapeutic and/or diagnostic procedures within the patient's body. For example, one or more instruments, (not shown), may be advanced through the delivery sheath 12 and into the vessel 94, alone or in conjunction with one another, as is known in the art. The one or more instruments may include catheters, e.g., balloon catheters, stent delivery catheters, imaging catheters, and the like; guidewires; filters; electrophysiology therapy and/or mapping devices; and the like. Thus, the procedures may include one or more of stent delivery, angioplasty, atherectomy, thrombectomy, angiography, cardiac mapping, ablation, and the like.

Upon completing the procedure(s), the instruments are removed from the introducer sheath, and the introducer sheath may be withdrawn at least partially, and preferably completely, from the puncture. Turning to FIG. 2D, once the introducer sheath is withdrawn, the sealing compound 99 (which may correspond to any of the embodiments described herein) surrounding the puncture 90 facilitates sealing and/or hemostasis within the puncture 90. For example, if the sealing compound 99 has gelled and/or solidified in the puncture 90 around the introducer sheath (not shown), the sealing compound 99 may be at least partially compressed between the introducer sheath and the surrounding tissue 96. In addition or alternatively, the vessel 94 proximal to the puncture site may be compressed to cease the blood flow before or while removing the introducer sheath.

When the introducer sheath is withdrawn from the puncture 90, the sealing compound 99 may expand inwardly into the puncture 90, thereby facilitating sealing and/or hemostasis. In addition or alternatively, the intervening tissue 96 surrounding the puncture 90 may at least partially recoil, further directing the sealing compound 99 into the puncture 90 to at least partially seal the puncture 90. If the sealing compound 99 is a lyophilized hydrogel or other material, e.g., in solid or powder form, the sealing compound 99 may be exposed to fluid, e.g., blood, from the vessel 94 after the introducer sheath is removed, causing the sealing compound 99 to hydrate further and/or swell, thereby further enhancing sealing the puncture 90.

Thus, as shown in FIG. 2D, sealing compound 99 may enhance hemostasis, thereby preventing substantial blood from escaping from puncture 90. Optionally, thereafter, external manual pressure may be applied to the skin 92 overlying the puncture 90 until complete hemostasis occurs. Preloading the sealing compound 99 in the puncture 90 may substantially reduce the time for hemostasis to occur, as compared to external pressure alone.

In alternative embodiments, one or more other sealing devices (not shown) may be introduced into the puncture 90 after the above-identified procedure(s) to further enhance hemostasis and/or sealing the puncture 90. For example, additional liquid sealing compound, e.g., hydrogel and/or hydrogel prepolymers, may be injected into the puncture 90 using the delivery sheath 12 or other devices. Exemplary apparatus and methods for sealing a puncture after a procedure are disclosed in above-incorporated application Ser. No. 10/454,362. Alternatively, other known sealing materials, e.g., plugs, clips, and the like, may be delivered into the puncture 90 after the procedure(s). Such sealing materials may include those that mechanically close a puncture, e.g., sutures, anchors, clips, those that promote clotting, e.g., thrombin, collagen, fibrin etc., and/or those that adhere, e.g., cyanoacrylates, fibrin glue, protein-based adhesives, synthetic adhesives, synthetic sealants, and the like. Thus, the preloaded sealing compound may be used to enhance sealing in addition to, or instead of, other known devices and methods.

In yet another alternative, a sealing compound may be introduced into the puncture 90 during the procedure, e.g., at any time after creation of the puncture 90 and/or before completion of the procedure. For example, an introducer sheath or other supplementary tubular member (not shown) may be advanced into the puncture 90 to at least partially fill the puncture 90 with sealing compound before removal of the final instruments, delivery sheath, and/or guidewire.

In addition to liquid hydrogel and/or precursor polymers, other sealing compounds may be delivered into the puncture 90 before accessing the vessel 94 to perform one or more procedures. For example, a solid hydrogel plug or powder may be delivered into the puncture 90, e.g., via the delivery sheath 12, shown in FIG. 1. Alternatively, other sealing materials, such as collagen or other hemostasis-promoting materials may be delivered into the puncture 90, as long as care is taken not to expose any thrombogenic materials within the vessel 94.

Turning to FIG. 6, an apparatus 310 is shown for delivering a plug of sealing material into a puncture (not shown) before accessing a vessel (also not shown) via the puncture to perform one or more medical procedures. Generally, the apparatus 310 includes a delivery sheath 312, which may be identical or similar to the delivery sheath 12 shown in FIG. 1 and described above. In addition, a sealing compound 399 is carried on an exterior of the delivery sheath 312 proximal to its distal tip 325.

The sealing compound 399 is an annular plug or other mass of lyophilized hydrogel, such as that disclosed in U.S. Pat. No. 6,605,294. The disclosures of this reference and any others cited therein are expressly incorporated herein by reference. The sealing compound 399 may be in a powder form, a hollow tube, or may be a solid mass, or rod. The sealing compound 399 may have a pre-delivery (e.g., pre-expanded or pre-swell state) diameter between about one and twenty five millimeters (1-25 mm), preferably between about five and ten millimeters (5-10 mm), and/or a length of between about five and twenty five millimeters (5-25 mm), preferably between about five and ten millimeters (5-10 mm). It will be appreciated by those skilled in the art that other shapes and/or configurations may be provided for the sealing compound 399.

Alternatively, other materials may be carried on the exterior of the delivery sheath 312 instead of a hydrogel, e.g., one or more biocompatible materials, such as collagen, thrombin, fibrin, polyglycolic acids (PGA's), polyactides (PLA's), and the like, which may be at least partially absorbed by the body over time.

Optionally, a cover 370 may be provided over the delivery sheath 312 that may at least partially cover the sealing compound 399. The sealing compound 399 may be pre-mounted to the delivery sheath 310 in its original pre-swelled size and/or squeezed or compressed into a smaller size/dimension in order to reduce its profile. The cover 370 is a relatively thin-walled sheath or peel-away skin, including a tapered distal tip 373 to facilitate atraumatic advancement through tissue. The cover 370 may be slidable relative to the delivery sheath 312, e.g., such that the cover 370 may be retracted to expose the sealing compound 199. Alternatively, the cover 370 may include one or more weakened regions (not shown) that may be separate when the cover 370 pulled proximally or apart to allow the cover 370 to be removed entirely from around the delivery sheath 312.

During use, the apparatus 310 may be introduced into a puncture (not shown), similar to the systems and methods described above for introducing the delivery sheath 12 shown in FIG. 1. The delivery sheath 312 may be introduced into the puncture, e.g., using a needle, guidewire, and/or other devices (not shown), as described above. With the distal end 324 of the delivery sheath 312 disposed within the vessel, the sealing compound 399 may be deposited within the puncture by moving the cover 370 to expose the sealing compound 399, e.g., by slidably retracting the cover 370 partially, or removing the cover 370 completely.

With the cover 370 retracted or removed, the sealing compound 399 is exposed within the puncture and/or to any fluid located within the puncture and/or surrounding tissue. For example, some fluid may be present naturally within the surrounding tissue that may at least partially hydrate the sealing compound, if the sealing compound is a lyophilized hydrogel. This would cause the sealing compound 399 to swell and/or expand within the puncture. Since the delivery sheath 312 is present, the sealing compound 399 may expand in size from the delivery sheath 312, thereby compressing the surrounding tissue.

A guidewire (not shown) may be introduced into the delivery sheath 310 (and/or through a needle used to create the puncture 90, not shown) to maintain access the body lumen 94. The delivery sheath 312 may then be removed from the puncture, and an introducer sheath (not shown) may be advanced into the puncture, similar to the embodiments described above, to perform one or more medical procedures. Alternatively, the delivery sheath 312 may be used as an introducer sheath, similar to embodiments described above. Once the medical procedure(s) is(are) performed, the introducer sheath (or delivery sheath 312) is removed from the puncture, leaving the sealing compound 399 behind. Optionally, the delivery sheath 312 may include a Teflon or other lubricious coating (not shown) on an exterior of the delivery sheath 312. The sealing compound 399 may be disposed over the coating such that the delivery sheath 312 may be slid relative to the sealing compound 399.

If the sealing compound 399 has not fully hydrated and/or expanded, it may expand further inwardly, thereby at least partially occluding the puncture. Thus, the sealing compound 399 may expand to many times, e.g., twice, three-times, or more, its pre-swelled diameter. If the sealing compound is fully hydrated, it may expand inwardly into the puncture or the surrounding tissue may recoil to further enhance sealing and/or hemostasis of the puncture, similar to the embodiments described above. If desired, external pressure may be applied and/or another sealing device may be delivered into the puncture, also similar to the embodiments described above.

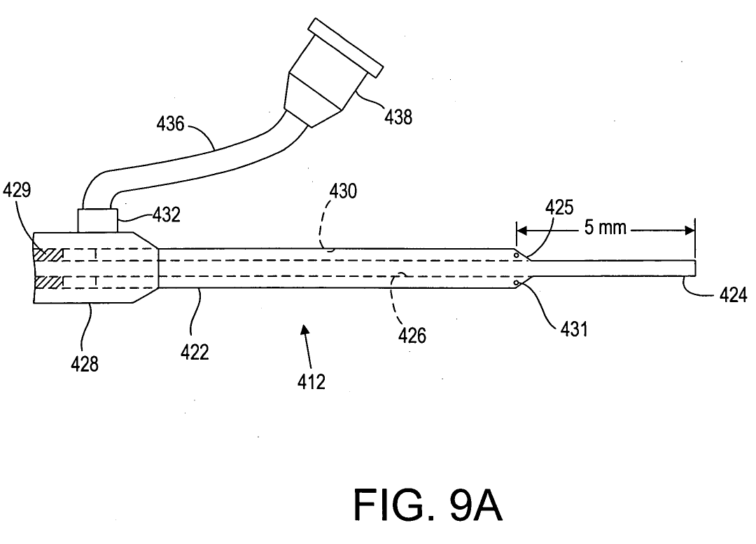

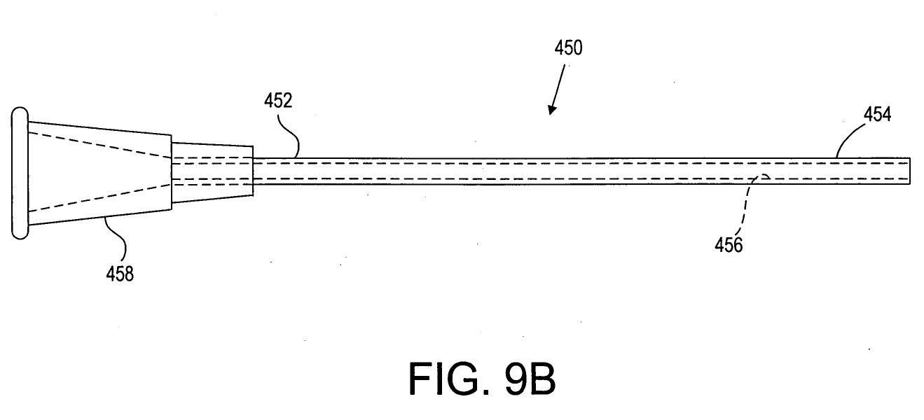

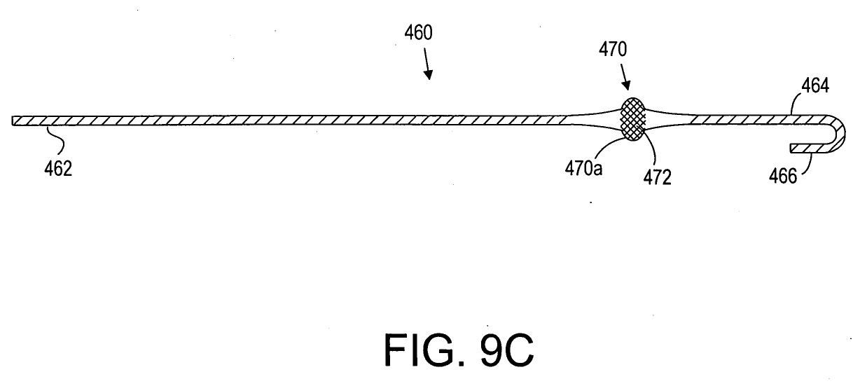

Turning to FIGS. 9A-9C, another embodiment of a system 410 is shown for sealing a puncture through tissue that generally includes a delivery or injection sheath 412, a retaining sheath 450, and a guidewire 460 having an expandable tamp 470 thereon. Optionally, the system 410 may include a needle, an introducer sheath, and/or a source of sealing compound, e.g., a dual syringe assembly (not shown), similar to the other embodiments described herein.

The delivery sheath 412 may be similar to the embodiments described elsewhere herein, e.g., including a proximal end 422, a distal end 424, a primary or guidewire lumen 426 extending between the proximal and distal ends 422, 424, and one or more secondary lumens 430. The delivery sheath 412 may include one or more outlets 431 in the wall of the delivery sheath 412, e.g., at a tapered intermediate portion 425 between the proximal and distal ends 422, 424. In an exemplary embodiment, two outlets 431 are located about five millimeters (5 mm) from the distal end 424.

Similar to the previous embodiments, a housing 428 may be attached to or otherwise provided on the proximal end 422 of the delivery sheath 412, including a side port 432 (or optionally multiple side ports, not shown) that communicates with an interior of the housing 428 and the secondary lumen 430. The housing 428 may include one or more seals 429 to seal the interior of the housing 428 such that sealing compound delivered via the side port 432 may be directed through the secondary lumen 430. The housing 428 may also include one or more seals (not shown), e.g., a hemostatic seal, for sealing the primary lumen 426. Tubing 436 may be connected to or otherwise extend from the side port 432 to a luer lock adapter 438, also similar to the other embodiments described herein.

Turning to FIG. 9B, the retaining sheath 450 may be an elongate tubular member including a proximal end 452, a distal end 454, and a lumen 456 extending therebetween. A hub 458 may be located on the proximal end 452, e.g., to facilitate manipulating the retaining sheath 450. The retaining sheath 450 may have a diameter or other size to allow the distal end 454 to be inserted into and/or through the lumen 426 of the delivery sheath 412, while the hub 458 may be larger than the size of the lumen 426, e.g., to provide a stop limiting distal advancement of the retaining sheath 450 into the delivery sheath 412. The retaining sheath 450 may be sufficiently flexible to conform to the surrounding anatomy, e.g., when the retaining sheath 450 is inserted into or removed from a puncture, e.g., along with other components, such as the guidewire 460.

Turning to FIG. 9C, the guidewire 460 may be an elongate member including a proximal end 462 and a distal end 464, e.g., including a “J” tip 466. The guidewire 460 may be formed from a solid wire, one or more coiled wires, and/or from a solid-walled tube, similar to conventional guidewires. The guidewire 460 may be formed from a variety of known materials, e.g., metals, such as stainless steel or Nitinol, plastics, and the like. Thus, the guidewire 460 may be sufficiently flexible to navigate tortuous anatomy, but may have sufficient column strength to be pushable from the proximal end 462.

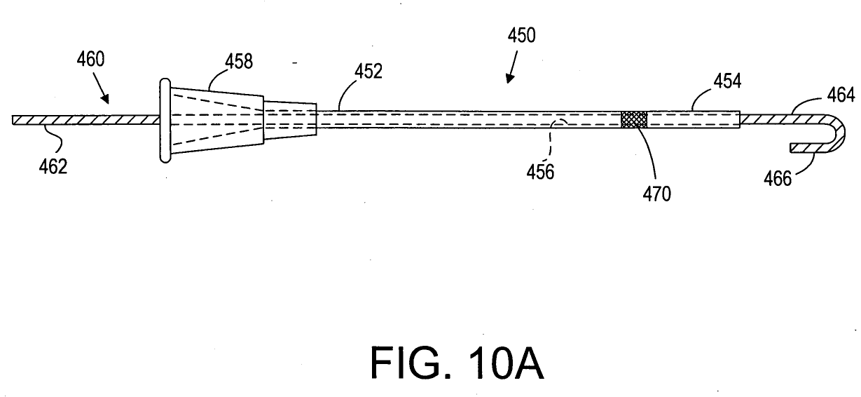

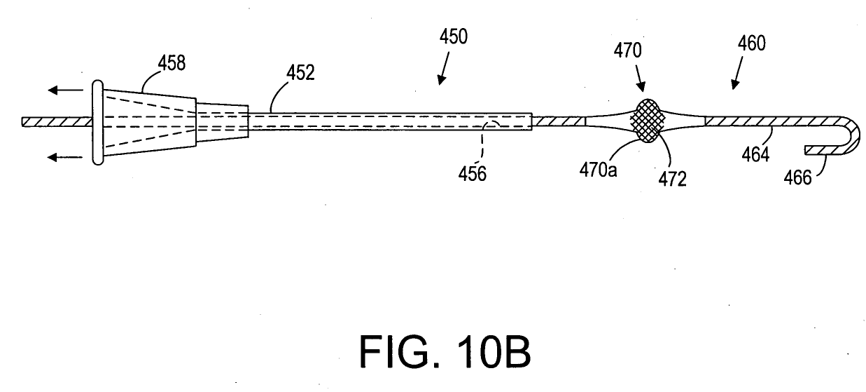

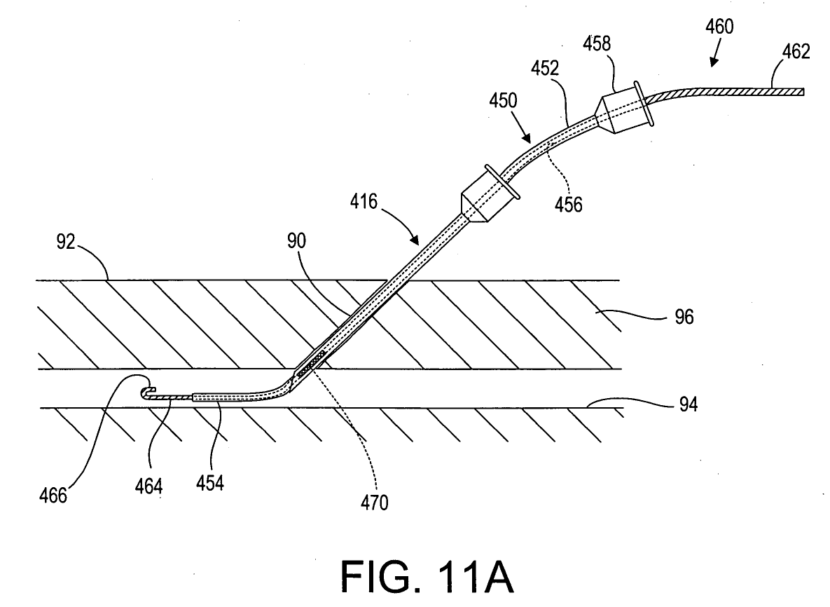

The tamp 470 may an expandable structure adjacent the distal tip 466 that may be biased towards an enlarged condition, as shown in FIGS. 9C and 10B, but may be resiliently compressible towards a contracted condition, as shown in FIG. 10A. In the embodiment shown in FIG. 9C, the tamp 470 includes a braided mesh of wires or other fibers 472 that assume a generally spherical or elliptical disk shape in the enlarged condition.

The fibers 472 may be formed from a shape memory material, e.g., Nitinol, stainless steel, a polymer or other plastic, and the like, that has the enlarged condition programmed into the fibers 472, e.g., by heat treatment. Thus, the fibers 472 may be elastically (or superelastically) deformed, e.g., compressed into the contracted condition using the retaining sheath 450, yet resiliently expand towards the enlarged condition once released, as explained further below. The tamp 470 may shorten as it expands from the contracted condition towards the enlarged condition, and may lengthen again as it is compressed back towards the contracted condition.

The fibers 472 may include a coating, cover, or other skin (not shown) that covers all or a portion of the tamp 470. For example, at least the proximal portion 470a of the tamp 470 may include a coating or other skin that extends across the spaces between the fibers 472 such that the proximal portion 470a is substantially nonporous. Alternatively, all of the tamp 470 may include a coating or other skin.

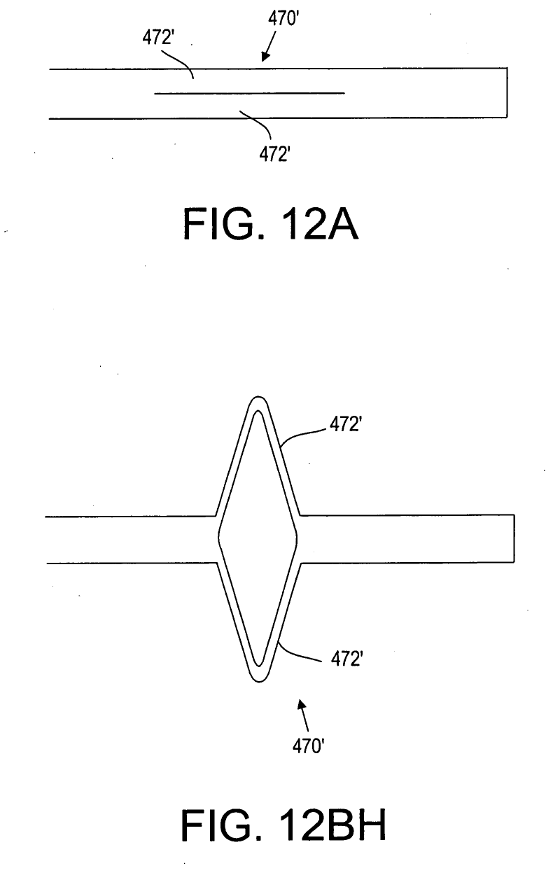

In a further alternative, as shown in FIGS. 12A and 12B, the tamp 470′ may include a plurality of struts 472′ that are expandable between enlarged and contracted conditions. The struts 472′ may extend substantially axially in the contracted condition and may buckle at an intermediate location thereon as they expand radially outwardly towards the enlarged condition. In one embodiment, the struts 472′ may be biased towards the enlarged condition, yet may be resiliently compressed towards the contracted condition, similar to the tamp 470 of FIG. 9C.

Alternatively, the struts 472′ may be selectively expanded and/or compressed, e.g., using an internal pull wire or other actuator (not shown). Exemplary embodiments of expandable strut and/or mesh structures, e.g., which may include coatings or skins, are disclosed in U.S. Pat. Nos. 6,238,412 and 6,635,068, and in application Ser. No. 10/143,514, published as Publication No. US 2003/0078616 A1. The entire disclosures of these references are expressly incorporated herein by reference.

Similar to the mesh tamp 470, the struts 472′ may include a coating or other skin, e.g., to extend between struts to provide at least a substantially nonporous proximal portion. In addition, the axial dimension of the struts 472′ may shorten as the struts 472′ move from the contracted condition to the enlarged condition, similar to the mesh tamp 470.

Turning to FIGS. 10A and 10B, the guidewire 460 of FIG. 9C may be inserted into the lumen 456 of the retaining sheath 450 of FIG. 9B. Thus, the retaining sheath 450 may be slidable axially along the guidewire 460, e.g., to selectively cover and uncover the tamp 470. When the retaining sheath 450 is directed over the tamp 470, as shown in FIG. 10A, the tamp 470 may be compressed towards and/or maintained in the contracted condition. When the retaining sheath 450 is retracted to expose the tamp 470, as shown in FIG. 10B, the tamp 470 may automatically expand towards the enlarged condition.

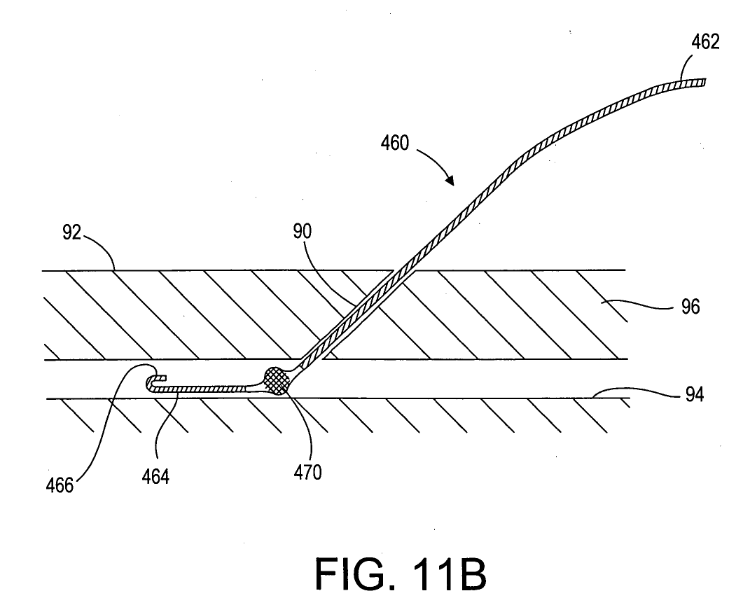

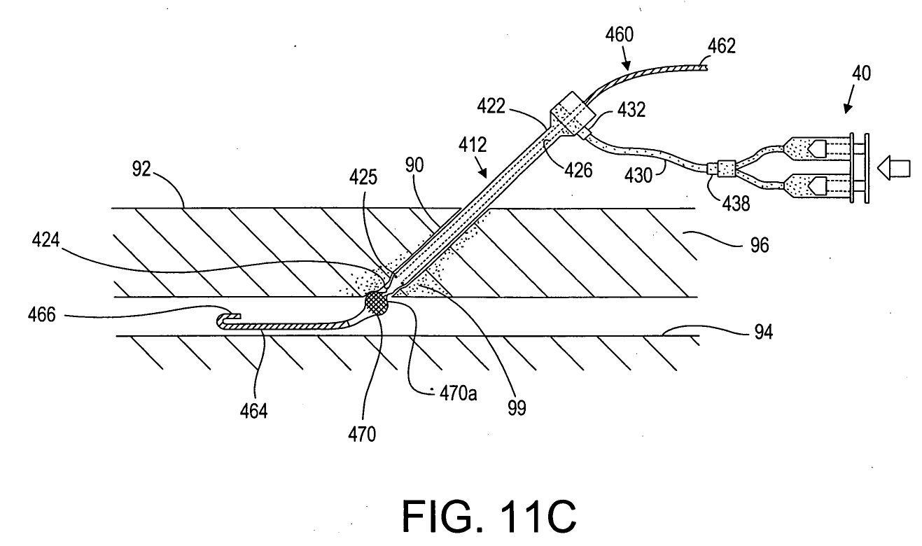

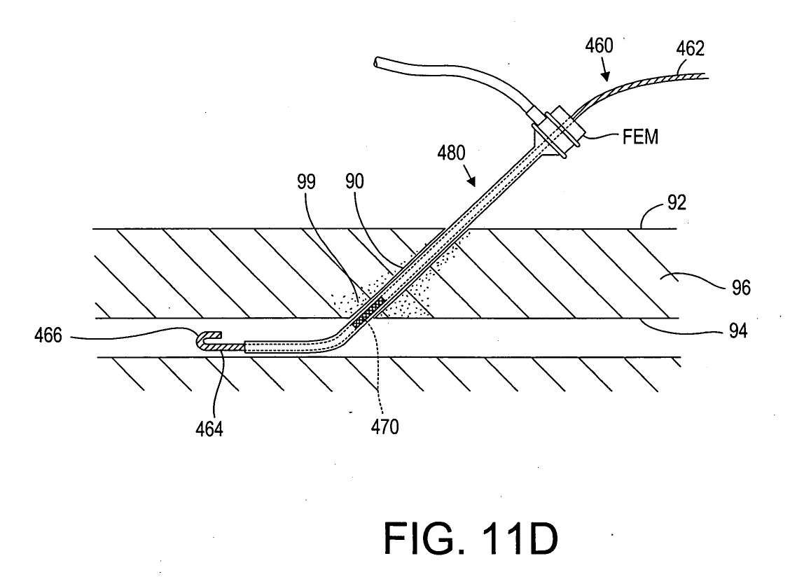

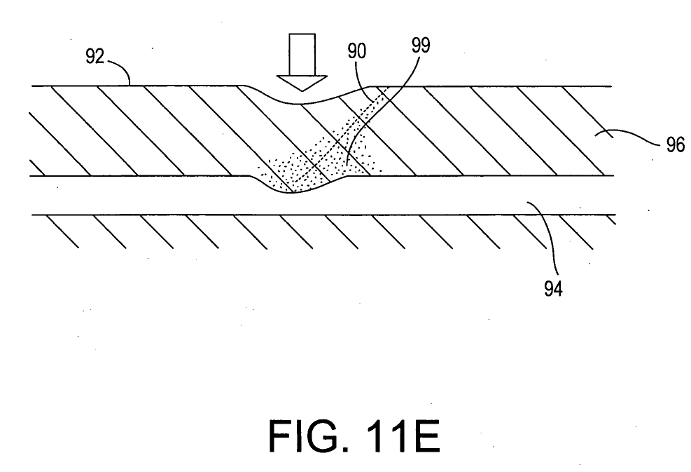

Turning to FIGS. 11A-11E, a method is shown for sealing a puncture 90 extending from a patient's skin 92 to a body lumen, e.g., a blood vessel 94, similar to other embodiments described elsewhere herein. Initially, as shown in FIG. 11A, a hollow needle 416 may be inserted through the patient's skin 92 to create the puncture 90 through intervening tissue 96, and into the vessel 94.

The guidewire 460 and retaining sheath 450 may be inserted into the puncture 90, e.g., through the needle 416 until the distal tip 466 is disposed within the vessel 94. As shown, the retaining sheath 450 covers the tamp 470 on the guidewire 460 as the guidewire 460 is advanced through the needle 416, thereby maintaining the tamp 470 in the contracted condition. Thus, the guidewire 460 may be advanced through the needle 416 until the tamp 470 is disposed within the vessel 94. Alternatively, it may be possible to compress the tamp 470 and insert it into the needle 416 without the retaining sheath 460.

Once the tamp 470 (e.g., covered by the retaining sheath 460) is within the vessel 94, the needle 416 may be removed, and the tamp 470 may be expanded within the vessel 94, as shown in FIG. 11B. For example, the retaining sheath 460 may be retracted at least partially, and/or entirely out of the puncture 90 to expose the tamp 470, whereupon the tamp 470 may automatically expand within the vessel 94. The hub 458 on the retaining sheath 450 may facilitate manipulation of the retaining sheath 450, e.g., during retraction to expose the tamp 470. Although not shown, the tamp 470 may shorten substantially as it expands to minimize occlusion of the vessel 94 when the tamp 470 is in the enlarged condition.

Alternatively, the tamp may be selectively expandable, e.g., using an internal pull wire or other actuator (not shown). Thus, once the tamp is exposed within the vessel 94, the tamp may be expanded, e.g., by pulling the pull wire until the tamp attains a desired enlarged size and/or configuration. In a further alternative, if the tamp is selectively expandable, it may be possible to eliminate the retaining sheath 460, e.g., to reduce the overall profile of the guidewire 460 during insertion into the puncture 90.

Turning to FIG. 11C, after the tamp 470 is expanded, the guidewire 460 may be partially retracted from the vessel 94 until the proximal portion 470a of the tamp 470 contacts the vessel wall and/or substantially seals the puncture 90 from the vessel 94. The delivery sheath 412 may be advanced over the guidewire 460, e.g., before or after the tamp 470 is expanded and/or retracted to seal the puncture 90. As shown, the delivery sheath 412 may be advanced over the guidewire 460 until the distal end 424 contacts the tamp 470, e.g., after the retaining sheath 450 is completely removed. For example, the proximal end 462 of the guidewire 460 may be backloaded through the primary lumen 426 of the delivery sheath 412, and the delivery sheath 412 may be advanced into the puncture 90, the guidewire 460 sliding through the primary lumen 426.

The outlets 425 of the delivery sheath 412 may be disposed a predetermined distance from the distal end 424 of the delivery sheath 412, e.g., at least about five millimeters (5 mm) such that the outlets 425 are disposed within the puncture 90 proximal to the vessel 94 when the distal end 424 contacts the tamp 470. When the distal end 424 contacts the proximal portion 470a of the tamp 470, tactile feedback may provide indication that the outlets 425 are located at the desired position for delivering sealing compound 99.

A source of sealing compound, e.g., dual syringe assembly 40, may be prepared and connected to the side port 432 of the delivery sheath 412 either before or after the delivery sheath 412 is advanced into the puncture 90, similar to the other embodiments described herein. The sealing compound 99 may then be delivered through the secondary lumen 430 and the outlets 425 and into the puncture 90. The sealing compound 99 may flow radially outwardly to permeate at least partially into the tissue surrounding the puncture 90. The delivery sheath 412 may be retracted as the sealing compound 99 is delivered, e.g., to fill the puncture 90 along its length.

Once the puncture 90 is sufficiently filled with the sealing compound 99, the guidewire 460 may be maintained such that the tamp 470 continues to seal the puncture 90 from the vessel 94, e.g., for sufficient time for the sealing compound 99 to at least partially or completely cure. Thereafter (or alternatively immediately after filling the puncture 90), the delivery sheath 412 may be removed entirely from the puncture 90.

Turning to FIG. 11D, an introducer sheath 480 may then be advanced over the guidewire 460 and into the puncture 90. The introducer sheath 480 may be a conventional introducer sheath similar to those described elsewhere herein, e.g., including a proximal end 482, a distal end 484, and a lumen 486 extending therebetween. The introducer sheath 480 may be advanced over the guidewire 460 until the distal end 484 contacts the tamp 470. Further advancement of the introducer sheath 480 while maintaining or retracting the guidewire 460 may cause the tamp 470 to compress to the contracted condition as it is directed into the lumen 486 of the introducer sheath 480. The guidewire 460 may then be removed entirely from the vessel 94 and puncture 90 by retracting the guidewire 460 through the lumen 486 of the introducer sheath 480.

Alternatively, after filling the puncture 90 with sealing compound 99, the delivery sheath 412 may be removed entirely from the puncture 90, leaving the guidewire 460 in place, e.g., to seal the puncture 90 from the vessel 94 while the sealing compound 99 gels or otherwise cures. Thereafter, the retaining sheath 450 may be advanced back over the tamp 470, e.g., by advancing the retaining sheath 450 over the guidewire 460 while maintaining the guidewire 460 within the vessel 94. This may cause the distal end 454 of the retaining sheath 450 to push against the tamp 470, causing the tamp 470 to compress towards the contracted condition as the tamp 470 enters the lumen 456 of the retaining sheath 450. The retaining sheath 450 and guidewire 460 may then be removed from the vessel 94 and puncture 90, e.g., after an introducer sheath 480 is advanced over the retaining sheath 450 and guidewire 460 into the puncture 90.

Once a distal end 484 of the introducer sheath 480 is disposed within the vessel 94, one or more instruments (not shown) may be advanced through the lumen 486 into the vessel 94, e.g., to perform one or more diagnostic and/or interventional procedures within the patient's body, as is known to those skilled in the art. Upon completing any such procedures, the instrument(s) may be removed from the vessel 94 through the introducer sheath 480.

As shown in FIG. 11E, the introducer sheath 480 may be removed from the vessel 94 and puncture 90, allowing the sealing compound 99 to substantially fill the puncture 90, thereby allowing and/or encouraging hemostasis to occur between the vessel 94 and puncture 90. Optionally, external pressure may be applied to the patient's skin 92 during removal of the introducer sheath 480, e.g., to further enhance sealing of the puncture 90 until hemostasis occurs.

While the invention is susceptible to various modifications, and alternative forms, specific examples thereof have been shown in the drawings and are herein described in detail. It should be understood, however, that the invention is not to be limited to the particular embodiments or methods disclosed, but to the contrary, the invention is to cover all modifications, equivalents and alternatives falling within the scope of the appended claims.

We claim:

1. A system for sealing a puncture extending through tissue to a body lumen of a patient, comprising:
an elongate member having proximal and distal ends with an expandable member on the distal end, the expandable member expandable from a contracted condition to an enlarged condition;
a delivery sheath having a proximal end, a distal end sized for insertion through the puncture, a primary lumen extending between the delivery sheath proximal and distal ends and sized for receiving the elongate member therethrough with the expandable member in the contracted condition, and a secondary lumen extending from a side port in the delivery sheath proximal end to one or more outlets located between the delivery sheath proximal and distal ends; and
a source of sealing compound coupled to the side port of the delivery sheath for delivering sealing compound through the secondary lumen and out the one or more outlets to tissue surrounding the puncture.

2. The system of claim 1, wherein the expandable member is biased towards the enlarged condition, the system further comprising a removable constraint for maintaining the expandable member in the contracted condition.

3. The system of claim 2, wherein the constraint comprises a tubular member slidable over the elongate member.

4. The system of claim 2, wherein the tubular member is slidable within the primary lumen of the delivery sheath such that the tubular member may be retracted through the primary lumen when the expandable member is disposed within the body lumen to allow the expandable member to expand within the body lumen.

5. The system of claim 1, wherein the expandable member comprises an expandable frame.

6. The system of claim 1, wherein the expandable member comprises a mesh structure.

7. The system of claim 1, wherein the expandable member comprises a plurality of struts.

8. The system of claim 1, wherein the expandable member comprises a substantially nonporous covering.

9. The system of claim 1, wherein the one or more outlets are disposed on a tapered portion of the delivery sheath.

10. The system of claim 9, wherein the delivery sheath comprises an extension between its tapered portion and distal end, the extension having a length greater than about five millimeters.

11. A method for sealing a puncture extending through tissue to a body lumen of a patient, comprising:
placing a guidewire through the puncture into the body lumen;
inserting a proximal portion of the guidewire into a primary lumen of a delivery sheath;
introducing the delivery sheath into the puncture by advancing the delivery sheath over the guidewire with the guidewire sliding within the primary lumen;
introducing a sealing compound into the puncture through one or more secondary lumens of the delivery sheath;
after introducing the sealing compound, accessing the body lumen through the puncture to perform a medical procedure via the body lumen.

12. The method of claim 11, wherein the sealing compound comprises a hydrogel.

13. The method of claim 11, wherein the sealing compound comprises a liquid.

14. The method of claim 11, wherein the sealing compound is delivered from one or more syringes into the one or more secondary lumens of the delivery sheath.

15. The method of claim 11, wherein the sealing compound is introduced by:
positioning the delivery sheath such that one or more outlets communicating with the one or more secondary lumens are located within intervening tissue surrounding the puncture; and
injecting the sealing compound through the one or more secondary lumens such that the sealing compound exits the one or more outlets and permeates at least partially into the intervening tissue.

16. The method of claim 15, wherein the delivery sheath is positioned such that the one or more outlets are disposed at least about five millimeters from the body lumen.

17. The method of claim 15, wherein the delivery sheath is positioned by monitoring blood flow from the body lumen into the one or more outlets and through the one or more secondary lumens to determine a relative location of the one or more outlets and the body lumen.

18. The method of claim 11, wherein the guidewire is placed through the puncture by:
inserting a sharpened elongate member through the patient's skin and intervening tissue and into the body lumen to create the puncture; and
advancing the guidewire through the sharpened elongate member and into the puncture.

19. The method of claim 11, wherein the body lumen is accessed by introducing one or more instruments through the tubular member into the body lumen.

20. The method of claim 11, further comprising withdrawing the delivery sheath from the puncture before accessing the body lumen through the puncture to perform the medical procedure.

21. The method of claim 11, wherein the guidewire comprises an expandable member on a distal end thereof, the guidewire being placed by advancing the distal end of the guidewire through the puncture with the expandable member in a contracted condition until the expandable member is disposed within the body lumen, the method further comprising:
expanding the expandable member within the body lumen;
retracting the guidewire with the expandable member expanded to substantially seal the puncture from the body lumen before introducing the sealing compound into the puncture.

22. The method of claim 21, further comprising collapsing the expandable member towards the contracted condition and withdrawing guidewire from the puncture.

23. A method for sealing a puncture extending through tissue to a body lumen of a patient, comprising:
advancing an elongate member carrying an expandable member in a contracted condition through the puncture until the expandable member is disposed within the body lumen;
expanding the expandable member within the body lumen;
retracting the elongate member until the expanded expandable member substantially seals the puncture from the body lumen;
advancing a delivery sheath over the elongate member into the puncture;
introducing a sealing compound into the puncture through the delivery sheath; and
after introducing the sealing compound, accessing the body lumen through the puncture to perform a medical procedure via the body lumen.

24. The method of claim 23, wherein the delivery sheath comprises a primary lumen, the elongate member sliding within the primary lumen as the delivery sheath is advanced into the puncture over the elongate member.

25. The method of claim 24, wherein the delivery sheath comprises one or more secondary lumens, and wherein the sealing compound is introduced into the puncture through the one or more secondary lumens.

26. The method of claim 25, wherein the delivery sheath comprises one or more outlets disposed proximal to a distal end of the delivery sheath, the one or more outlets communicating with at least one of the one or more secondary lumens for delivering the sealing compound introduced through the one or more secondary lumens into tissue surrounding the puncture.

27. The method of claim 26, wherein the one or more outlets are disposed a predetermined distance from the expandable member when the sealing compound is delivered into the puncture.

28. The method of claim 27, wherein the delivery sheath is advanced into the puncture until the distal end of the delivery sheath contacts the expandable member, thereby providing tactile feedback that the one or more outlets are disposed at the predetermined distance.

29. The method of claim 23, further comprising collapsing the expandable member towards the contracted condition and withdrawing the elongate member from the puncture.

30. The method of claim 29, wherein the body lumen is accessed by advancing an introducer sheath over the elongate member into the puncture until a distal end of the introducer sheath is disposed within the body lumen.

31. The method of claim 30, wherein the elongate member is withdrawn through the introducer sheath, thereby causing the expandable member to collapse as it enters the primary lumen.

32. The method of claim 29, wherein the expandable member is collapsed by directing a tubular member over the elongate member.

33. The method of claim 23, wherein the expandable member is biased towards an enlarged condition, and wherein the expandable member is expanded by removing a constraint overlying the expandable member, whereupon the expandable automatically expands towards the enlarged condition.

34. The method of claim 23, wherein the expandable member is expanded by activating an actuator on a proximal end of the elongate member.

35. A method for delivering sealing compound into a puncture extending through tissue between a patient's skin and a body lumen, the method comprising:
advancing a needle 116 into the patient's skin to create the puncture and advancing the needle until a distal tip of the needle enters the body lumen;
advancing a guidewire through the needle into the body lumen;
withdrawing the needle at least partially from the puncture to withdraw the distal tip a predetermined distance from the body lumen; and
delivering sealing compound into the puncture through the needle.

36. The method of claim 35, wherein the needle is advanced until bleed back provides a visual indication that the distal tip is located within the body lumen.

37. The method of claim 35, wherein the predetermined distance that the distal tip of the needle is withdrawn from the body lumen is between about five and ten millimeters (5-10 mm).

38. The method of claim 35, further comprising:
advancing a guide element along the needle after the distal tip is disposed within the body lumen until the guide element contacts the patient's skin; and
maintaining the guide element against the skin while the needle is withdrawn to provide a visual indication of the distance that the needle and the distal tip 166 are withdrawn relative to the body lumen.

39. The method of claim 35, wherein sealing compound is delivered by:
coupling a fitting to a proximal end of the needle to allow a source of sealing compound to communicate with a lumen extending through the needle between the proximal end and the distal tip.

40. The method of claim 39, wherein the fitting comprises a “Y” adapter including a distal end that is coupled to the proximal end of the needle, a first proximal end through which the guidewire extends, and a second proximal end to which the source of sealing compound is connected.

41. The method of claim 35, further comprising at least partially withdrawing the needle as the sealing compound 99 is delivered to fill at least a portion of the puncture with the sealing compound.

42. The method of claim 35, further comprising removing the needle entirely from the puncture, leaving the guidewire in place.

43. The method of claim 42, wherein compression is applied on the patient's skin to secure the guidewire within the puncture while the needle is removed.

44. The method of claim 42, further comprising introducing an introducer sheath into the puncture over the guidewire after the needle is removed to access the body lumen.
